(19) United States
(12) Patent Application Publication
(10) Pub. No.: US 2026/0130774 A1
(43) Pub. Date: May 14, 2026

(54) SHAPED ARTICLE

(71) Applicant: ACADEMISCH ZIEKENHUIS LEIDEN (H.O.D.N. LUMC), Leiden (NL)

(72) Inventors: Bart Godefridus Catharina Wilhelmus PIJLS, Leiden (NL); Richard VAN DE WATERINGEN, Leiden (NL)

(73) Assignee: ACADEMISCH ZIEKENHUIS LEIDEN (H.O.D.N. LUMC), Leiden (NL)

(21) Appl. No.: 18/704,293

(22) PCT Filed: Nov. 22, 2022
(86) PCT No.: PCT/NL2022/050670

§ 371 Date: Apr. 24, 2024

(30) Foreign Application Priority Data
Nov. 23, 2021 (NL) 2029879

(51) Int. Cl. A61F 2/46; A61F 2/30; A61F 2/38

(52) U.S. Cl. CPC A61F 2/4657; A61F 2/3859; A61F 2/389; A61F 2002/30604; A61F 2002/30617; A61F 2002/30668; A61F 2002/30677; A61F 2002/465; A61F 2002/4672

(57) ABSTRACT

The present invention concerns a shaped article sized and dimensioned to at least partly and removably cover a prosthetic or implant, wherein said shaped article comprises means for monitoring a temperature change in said article.

### FIELD OF THE INVENTION

The present relates to an article that is configured to at least partly and removably cover a prosthetic or implant. In particular, but not exclusively, the present invention relates to a shaped article that can be used in a process of inductive heating of prosthetics or implants, or at least portions thereof, such as for treating biofilm infections of metal implants.

### BACKGROUND OF THE INVENTION

Infection of implants is a major problem in elective and trauma surgery. A considerable proportion of prosthetic implants and an even greater proportion of implants in fractures become infected.

Such infections can trigger an immune response that can at least help counter the infection, particularly in combination with antibiotics and/or other drugs. However, especially where an undesirable biofilm develops at or on the prosthetic, treatment is often by invasive surgery. The infected area is opened up, cleansed and treated. To eradicate the infection effectively, multiple surgeries and long term antibiotic treatment are sometime needed, often causing great discomfort to the patient. Some prior art procedures disclose heating of limbs or patients comprising implants, however requiring very high field strengths, and likely leading to internal burns due to the difficult control of the heat deployment.

For example, where a hip prosthetic is associated with an infection, an existing (or new) wound is opened up to access the hip prosthetic. The hip prosthetic is exposed to allow thorough cleansing of the prosthetic and surrounding tissue to try to eradicate the infection. In some cases, the infection may necessitate the removal of the prosthetic, such as for replacement by a new prosthetic.

US-A-2011/251687 discloses a small coil implement to heat small oral shape memory material implant by induction for a period of less than 5 seconds, to return it to its original shape after introduction into a human ear. However, the size of the coil does not permit to generate a field strong enough to heat an implant or prosthetic to reduce a biofilm on a surface thereof, as exemplified in the data relating to the eddy current penetration depth in Nitinol summarised herein. WO2018/013935 discloses a method and apparatus for heating implants, by submitting the entire body of patient to a magnetic field. While this method can heat an implant, the control of the field and energy supplied is difficult to dose or concentrate at a specific area, resulting in enhanced tissue damage possibly leading to loss of fixation and subsequent revision surgery. Also, in presence of an infection, the heat generated may be dissipated quickly by tissue and surrounding liquids, and hence renders the disclosed method unsuitable. Furthermore, adequate treatment requires the identification of the responsible microorganism and its susceptibility to antibiotics, which normally requires several (5-6 tissue) cultures; this is not possible with a non-invasive method.

Recently, the present applicant has developed an apparatus for heating at least a portion of a prosthetic or implant. Such an apparatus may comprise a coil, the coil being configured to inductively heat the portion of the prosthetic or implant. Such an apparatus may be configured to heat the portion of the prosthetic or implant to a temperature at or below a maximum temperature, the maximum temperature being associated with tissue damage; and the apparatus is configured to heat the portion of the prosthetic or implant to a temperature at or below a minimum temperature, the minimum temperature being associated with infection control.

It would be desirable to provide further ways of heating prostheses or implants by means of inductive heating, for example by more targeted or more stable heating. It would also be desirable to provide means for determining whether a certain pre-set temperature has been reached as a result of heating a prosthetic or implant. For example, it would be desirable for a surgeon to have visual feedback during surgery regarding the temperature or temperature distribution of an implant.

It may be an object of one or more aspects, examples, embodiments or claims of the present disclosure to solve or at least mitigate one or more of the shortcomings of the prior art, such as herein described or otherwise.

### SUMMARY OF THE INVENTION

In a first aspect of the present disclosure, there is provided a shaped article sized and dimensioned to at least partly and removably cover a prosthetic or implant, wherein said shaped article comprises means for monitoring a temperature change in said article.

The article according to the present disclosure is particularly suited for use in a method of inductively heating at least a portion of a prosthetic or implant, for example during joint replacement surgery. The article is of such size and shape that it can at least partially cover a prosthetic or implant. The article according to the present disclosure is furthermore of such size and shape that it can be removed from the prosthetic or implant, for example after a certain pre-set temperature of at least a part of the prosthetic or implant has been reached due to heating of at least a part of the prosthetic or implant.

Such a method of inductively heating at least a portion of a prosthetic or implant is disclosed in WO 2020/067898 A2 as well as its priority application NL 2 021 722 B1. Herein, an apparatus and a method for inductively heating at least a portion of a prosthetic or implant is described, wherein the apparatus comprises an external induction coil, the external induction coil being external to a body or housing of the apparatus. The coil is sized and dimensioned to be smaller than a body portion associated with the prosthetic or implant. In operation, a portion of a prosthetic or implant may be positioned within a distance of the coil and an administration of heat may be controlled by controlling a power, such as an amount and/or duration of power, supplied to the induction coil. The apparatus of WO 2020/067898 A2 may comprise or be in data communication with a temperature measurement device, such as a thermal sensor, camera or thermometer integrally comprised in the heating apparatus. While no further details are provided on that point, it is evident that the monitoring of a temperature of the device and or of the coil itself are not relevant information in this regard.

The article according to the present disclosure comprises means to monitor a change of temperature in the article. In other words, the present article configured to removably cover at least a portion of a prosthetic or implant allows monitoring temperature changes in the article itself. Since the shaped article is sized and dimensioned to cover at least a portion of a prosthetic or implant, a temperature of the prosthetic or implant will closely correspond to the temperature of the shaped article, in particular where the surface of the prosthesis is, preferably substantially or fully, covered with the article. By having such means for monitoring a change of temperature for example embedded in, or attached to the shaped article of the present disclosure, temperature measurement and/or temperature control of the prosthetic or implant is possible without the need for expensive equipment such as thermal cameras and without damaging or scratching the prosthetic or implant itself, for example due to the application of one or more external temperature sensors. Furthermore, the presence of such a means to monitor a change of temperature in the object also allows for selectively heating a single portion of the prosthetic or implant at a time.

Since the article according to the present disclosure is sized and dimensioned to at least partly cover a prosthetic or implant, the article may advantageously also provide a thermal barrier for any surrounding tissue during heating, thus reducing the thermal dose to this surrounding tissue.

Another advantage of the shaped article according to the present disclosure is that it allows for the inclusion and/or attachment of one or more coils for induction heating, which in turn permits these one or more induction coils to be maintained at a constant distance of the implant or prosthetic during induction heating. Furthermore, it allows for targeting only the portion of the prosthetic or implant to be heated, such as with a precision coil, and/or for the creation of a specific temperature gradient in the prosthetic or implant. It is apparent that the article according to the invention does not consist of a coil as such but merely may comprise it, and hence the temperature change in the article concerns the body material of the article rather than the coil(s) and/or any apparatus to which the coil is connected.

In another aspect the present disclosure there is provided a method of inductively heating at least a portion of a prosthetic or implant, the method comprising
covering at least portion a prosthetic or implant with the shaped article according to the present disclosure; and
inductively heating the portion of the prosthetic or implant using one or more induction coils.

By using the shaped article according to the present disclosure to cover at least portion a prosthetic or implant during the inductive heating procedure, it is possible to locally and precisely heat one or more selected parts of the prosthetic or implant, without damaging or scratching the prosthetic or implant itself and without inflicting unnecessary thermal or other damage to the surrounding tissue.

### BRIEF DESCRIPTION OF THE DRAWINGS

FIG. 1 shows an example of a shaped article;

FIG. 2 shows an example of a shaped article;

FIG. 3 shows a shaped article according to an embodiment of the present disclosure;

FIG. 4 shows a metal implant, and a metal implant partially covered with a shaped article according to an embodiment of the present disclosure;

FIG. 5 shows an example of a shaped article;

FIG. 6 shows a shaped article according to an embodiment of the present disclosure;

FIG. 7 shows shaped article according to an embodiment of the present disclosure, and a thermal image indicative of selective heating of a portion of the article.

### DETAILED DESCRIPTION OF THE INVENTION

Accordingly, one aspect of the present disclosure relates to a shaped article sized and dimensioned to at least partly and removably cover a prosthetic or implant, wherein said shaped article comprises means for monitoring a temperature change in said article.

The term “prosthetic or implant” herein may relate to orthopaedic implants such as for instance a total joint replacement, a total or hemi-joint arthroplasty; a total joint prosthetic; a joint replacement; e.g. a hip prosthetic; a joint replacement implant; joint prosthetic; joint reconstruction prosthetic; a joint reconstruction implant; a fracture fixation device, including but not limited to plate, screws, nails, staple, rods, wire, pins, external fixators, such as for clavicle, spine, ankle, hip, tibia, or femur fractures; a joint distraction device, i.e. an external fixator, e.g. for treatment of fractures or for knee distraction; mega implants after tumour resection, e.g. acetabular cup; and dental implants and prostheses. Preferably, the term “prosthetic or implant” refers to a loaded or loadable implant. As an example, during joint replacement surgery, also referred to as replacement arthroplasty, a joint implant is inserted into or otherwise attached to a bone that has been prepared to receive the implant, and the implant is secured to achieve a reliable stabilization, or fixation, for bearing loads. Accordingly, the term “prosthetic implant” herein preferably refers to a (partial) metal device with biomechanical function(s) implanted near, to or in a bone.

As used herein, the term “invasive species” relates to certain invasive microorganisms, in particular bacteria, to form colonies, and eventually a biofilm. A biofilm typically comprises a conglomerate of microbial organisms embedded in a highly hydrated matrix of exopolymers, typically polysaccharides and/or other macromolecules. Biofilms may contain either single or multiple microbial species and readily adhere to surfaces of prosthetics and/or implants. Prevention of colonization by, and eradication of biofilm-associated microorganisms is often difficult, as the structural matrix established during biofilm formation usually allows the colonizing cells to withstand normal treatment doses of antibiotics, as the glycocalyx matrix appears to serve as a barrier protecting and isolating the microorganisms from host defence mechanisms such as antibodies and phagocytes as well as from antimicrobial agents including surfactants, biocides and antibiotics. Once established, a biofilm itself may be refractory to treatment, providing, in effect, a reservoir of infection which can lead to recurrence of the infection after an antibiotic treatment.

Biofilm infection is often associated with septic arthritis, where biofilm formation on joint or implant surfaces can lead to a chronic and recurrent infection. In addition to sepsis, the biofilm infection can cause destruction of the joint surface material, in particular in implants, such as bone prostheses and the like, thereby necessitating additional surgery or surgeries, commonly referred to as corrective or revision surgeries to remove the infectious material, as well as necrotic or infected tissue.

The term “active count” herein relates to a measurement for the determination of active or viable invasive microorganisms in or derived from a biofilm after the treatment. This may for instance be done by a probe active count assay (PAC), or by a different cell count method or protocol.

The shaped article is sized and dimensioned to at least partly cover a prosthetic or implant. As such, the article has a general size and dimensions substantially corresponding to the known dimensions of customary implants and prostheses for application in the human and animal body, for example as described in the preceding paragraph. For example, the article may have dimensions (measurements) in all dimensions (length, width, height) substantially the same as those of a given prosthetic or implant, or it may have one or more dimensions, for example a length, that are smaller or larger than the corresponding length of a given prosthetic or implant. Typically, such a dimension that is smaller or larger than the corresponding length of a given prosthetic or implant, does not deviate by more than 50%, preferably more than 40%, more preferably more than 30%, 20% or 10% from the corresponding length of a given prosthetic or implant. The general size and dimensions of the shaped articles may be further determined by the specific shape of a certain class or type of prosthetics or implants. Examples of classes of prosthetics or implants are those used in joint replacement surgery, such as total knee replacement (TKR) or total knee arthroplasty (TKA), partial knee replacement, partial or total shoulder replacement surgery (glenoid component and humeral (stem) component), ankle replacement (talar component and tibia component), as well as those employed in spine, hip, elbow, wrist, finger and thumb replacement surgery. Other examples include external fixator pins, e.g. for treatment of fractures (see e.g. https://en.wikipedia.org/wiki/External_fixation) or for knee distraction, mega implants after tumour resection (see e.g. http://implantcast-na.com/lumic-pedestal-cup/) and dental implants and prostheses. Examples of specific shapes of such prosthetics and implants can be found in publicly accessible online databases such as https://orthoload.com and https:/www.odep.org.uk/products.aspx. In one embodiment, the orthopaedic implant, prosthetic or device is a customised implant, prosthetic or device. In one embodiment, the shaped article is dimensioned and formed such that it allows covering a customised implant, prosthetic or device and/or allows further customisation of its dimensions and/or form just before, or during surgery, for example by cutting or tearing.

The orthopaedic implant, prosthetic or device may also be a plate, such as a bridging plate that is used in the stabilization or fixation of complex multi-fragmentary fractures, wherein it is necessary to minimize stripping soft tissues of the intermediate fragments.

In an embodiment, the article has a rigid or semi-rigid shape that is dimensioned to follow a contour of an exterior surface of a prosthetic or implant. In another embodiment, the article is flexible and can be shaped or deformed to follow a contour of an exterior surface of a prosthetic or implant. The contour of an exterior surface of the prosthetic or implant may be followed partially, or it may be followed substantially entirely.

Examples of rigid or semi-rigid shapes are a mould, cup, half-open tube, or a concave hemisphere. Examples of flexible shapes are a sheet or a slab of a flexible material, which is conveniently shaped over or around the prosthetic or implant. Semi-rigid or flexible shapes may advantageously allow placement during a surgical procedure through a surgical incision. Combinations of one or more rigid or semi-rigid sections and one or more flexible sections within a single article according to the present disclosure are also possible. In an embodiment, the shaped article is or comprises a sheet or a slab made of a flexible material from which a desired shape can be cut out or carved out prior to at least partially covering the prosthetic or implant with said cut out or carved out shape.

Preferably, the article has dimensions such that it covers at least 20%, preferably at least 30%, or at least 40%, more preferably at least 50%, 60%, 70%, 80%, or 90%, or substantially all of the total exterior surface of a prosthetic or implant.

Typically, the article has dimensions such that in at least one direction it has a length between 10 mm and 300 mm, preferably between 20 and 200 mm, more preferably between 15 and 150 mm, even more preferably between 20 and 100 mm, or between 30 and 50 mm.

In some embodiments, the body tissue that is temporarily repositioned during surgery, for example the tendon and patella in knee surgery, may aid in ensuring that he shaped article is held in place, for example by exerting pressure on the article.

The shaped article is sized and dimensioned to removably cover at least a portion of a prosthetic or implant, i.e. the shaped article should capable of being removed from the prosthetic or implant without undesirable fracture, disintegration, irreversible deformation, leaving debris, damage (such as scratches), etc on the implant or prosthetic.

It follows from the above that the term “shaped” in “shaped article” is to be interpreted in the context of the prosthesis or implant to which the article of the invention is applied. In other words, depending on the dimensions and shape of the prosthesis or implant in question, as well as the extent to which it is to be covered by the article for the purpose of monitoring temperature change in said prosthesis or implant (and subsequent removal of the article therefrom), the skilled person will be able to choose a suitable form and corresponding dimensions.

In an embodiment, the article is sized and dimensioned so as to generically at least partly cover multiple shape variants within a class of well-known and commonly available prostheses or implants. For example, the article may be sized and dimensioned so as to generically at least partly cover common metal tibial components or femoral component of a knee replacement surgery, talar or tibial component of ankle replacement surgery, glenoid component or humeral (stem) component of partial or total shoulder replacement surgery, acetabular or hip femoral stem implant components, etc., or common bone fixation plates for, for example, clavicle, ankle, hip or femur trauma.

In another embodiment, the article is sized and dimensioned so as to at least partly cover custom-made implants, for example in the form of custom-made mould. Such a mould may be further adapted or customised just before or during surgery, for example by cutting or modelling into a desired shape.

The shaped article comprises means for monitoring a temperature change in said article. The temperature change may be the attainment of a predetermined temperature. The temperature change can be the achievement of a predetermined temperature difference based on a starting temperature, so that it corresponds to a specific thermal dose of thermal accumulated in the implant or prosthetic.

The means of monitoring a temperature change may be provided be the presence of one or more temperature sensors or thermal sensors. Such temperature sensors or thermal sensors may be attached externally to the shaped article or they may be at least partially embedded in the shaped article. A combination of such sensors attached externally and at least partially embedded in the shaped article to the shaped article is also possible. In an embodiment, the shaped article itself holds the sensors in place during the surgical procedure. Preferably, the one or more temperature sensors or thermal sensors are substantially fully or fully embedded in the shaped article. The temperature sensors or thermal sensors may be e.g. thermistors, thermocouples, fibre optics or a combination thereof. The one or more temperature sensors or thermal sensors may be connected to an apparatus comprising a temperature control mechanism. The temperature control mechanism may be part of or incorporated into or linked to a heating apparatus, preferably an inductive heating apparatus.

The means of monitoring a temperature change may be provided be the presence in or applied on, or a combination thereof, of a material that is capable of providing a visual indication of whether a predetermined temperature or temperature change has been reached in at least a portion of said article. In an embodiment, such a material that is capable of providing a visual indication of whether a predetermined temperature or temperature change has been reached is a material that exhibits a change in optical transmittance, a change in optical reflectance, a change in luminescence (fluorescence and phosphorescence), or a combination thereof in response to a temperature change. Such a change in optical transmittance, optical reflectance, luminescence, or a combination thereof, as result of a temperature change is typically perceived by the human eye as a change in the colour of the article or a part of the article. Thus, in an embodiment of the present disclosure, the shaped article comprises a material that changes colour in response to a temperature change. As such, when the article is used to, partially or substantially fully, cover a prosthetic or implant, such that it is in close contact with the prosthetic or implant, and when the prosthetic or implant is heated using inductive heating, the colour change may provide a visual indicator of whether (parts of) the prosthetic or implant have reached a pre-set temperature or whether a pre-set amount of thermal energy has been accumulated. Accordingly, the use, in or on the shaped article, of a material that is capable of providing a visual indication of whether a predetermined temperature or temperature change has been reached provides the operator, such as a surgeon, with a means for monitoring a temperature change in the article, which by virtue of the proximity of the prosthetic or implant, closely represents a temperature change of the prosthetic or implant, typically the surface of the prosthetic or implant.

In some embodiments, the change in optical transmittance, reflectance, luminescence or a combination thereof in response to a temperature change may not be visible to the human eye (for example, it is temperature-responsive at ultraviolet or infrared wavelengths), but may be registered by suitable optical detection equipment and converted to a visible signal, thus providing an indirect signal to a human operator.

The material that is capable of providing a visual indication of whether a predetermined temperature or temperature change has been reached in at least a portion of the shaped article may be a thermochromic pigment, liquid crystal, paint, polymer, ink, or dye, or a combination thereof, or any other material that can be embedded in or coated on the article, or combinations thereof.

Examples of such thermochromic materials are inorganic compounds such as (transition) metal oxides and metal carbonates, organic cholesteric liquid crystals, organic donor-acceptor compounds, etc., examples for which can be found in e.g., EP3856855A1; U.S. Pat. No. 5,558,700A; WO2015048536A1; Cheng, H., Yoon, J., & Tian, H. (2018), Recent advances in the use of photochromic dyes for photocontrol in biomedicine. Coordination Chemistry Reviews, 372, 66-84; Seeboth, A., Lötzsch, D., Ruhmann, R., & Muehling, O. (2014), Thermochromic Polymers-Function by Design. Chem. Rev., 114(5), 3037-3068; Jesse H. Day, Thermochromism of inorganic compounds, Chem. Rev., 68(6), 649-657. Other examples are organic or inorganic photonic coatings having a periodic nanostructure that display a tuneable colour-changing response.

The temperature-responsive materials may be colourless at ambient temperature and only become coloured after a pre-set increase in temperature, which is characteristic for so-called leuco dyes. They may also switch between two distinct colours upon a temperature change, for example between dark green for ambient temperature and light green to white for a temperature increase to 60° C.

The material that is capable of providing a visual indication, typically a colour change, of whether a predetermined temperature or temperature change has been reached in at least a portion of the shaped article may be embedded in the shaped article, or provided as a coating on the shaped article, or a combination thereof. For example, a thermochromic pigment may be dispersed through the bulk composition, e.g. a silicon polymer, of the shaped article in a sufficient concentration to provide the desired change in optical transmittance or reflectance, or a combination thereof, upon a pre-determined temperature change of the prosthetic or implant that is (fully or partially) covered by said shaped article. The thermochromic material may be encapsulated in a suitable encapsulating agent or be used as a such.

The change in optical transmittance, optical reflectance, luminescence, or a combination thereof in response to a temperature change may be a reversible change. The change in optical transmittance, optical reflectance, luminescence, or a combination thereof in response to a temperature change may be an irreversible change. Combinations of reversible and irreversible optical changes over a certain temperature trajectory are also possible. For example, a reversible change (e.g. from dark green to transparent) may occur during active heating, followed by an irreversible change (e.g. from transparent to white) when the target temperature has been reached). The thermochromic effect may also be of a “memory effect” nature, wherein a reversible colour change occurs at a fist temperature T1 and returns to its original colour when the temperature drops to second temperature T2. Thus, a (partly) irreversible optical change in the shaped article of the present invention, for example by an irreversible thermochromic dye, may be utilized for visualizing if or to which extent a particular surface, or part of a surface, has reached a target temperature. This may, for example, aid a surgeon in keeping track which part of the metal surface of an implant or prosthetic has been adequately treated and which part of the surface still needs to be heated.

It is also possible that the shaped article contains a combination of one or more thermal sensors as described above with a material that can provide a visual indication of a temperature change.

The temperature change to be monitored with the shaped article of the present disclosure should correspond to a temperature in the prosthetic or implant, or a portion thereof, in the proximity of the position of the shaped article, wherein said temperature is associated with a desired temperature-related effect in that prosthetic or implant, or in a portion thereof. For example, the desired temperature-related effect may be associated with infection control. For example, a certain minimum temperature to be reached in at least a portion of the prosthetic or implant may be associated with killing, or at least combatting, infection. In at least some examples, the minimum temperature may be at least 38° C.; at least 40° C.; at least 50° C.; at least 60° C.; at least 65° C.; at least 70° C.; or at least 75° C.; or at least 80° C.; or at least 90° C. The maximum temperature may be at least 60° C.; at least 65° C.; at least 70° C.; at least 75° C.; or at least 80° C.; or at least 90° C.; or at least 100° C.; or at least 110° C. or at least 120° C. Preferably, the prosthetic or implant, or a portion thereof, is heated to a temperature from about 60° C. to about 90° C., in particular of from 65° C. to 70° C., preferably for a period suitable to reduce the active count of invasive species forming a biofilm.

Since the article according to current disclosure is shaped in such a way that it preferably closely covers the prosthesis, the temperature difference between the covered part of the prosthetic and the article in the region of overlap will be small. In any case, a skilled person will be able to derive a relation between the temperature reached in the prosthetic or implant and the temperature to be monitored in the shaped article as described herein. Accordingly, in at least some examples, the temperature change to be monitored in the article of the present disclosure may be at least 38° C.; at least 40° C.; at least 50° C.; at least 60° C.; at least 65° C.; at least 70° C.; or at least 75° C.; or at least 80° C.; or at least 90° C. The maximum temperature may be at least 60° C.; at least 65° C.; at least 70° C.; at least 75° C.; or at least 80° C.; or at least 90° C.; or at least 100° C.; or at least 110° C. or at least 120° C. Depending on the selected lower and/or upper limits of the temperature to be monitored for a particular purpose, the skilled person will be capable of selecting appropriate means for monitoring said temperature value(s). For example, when a thermochromic pigment embedded in the shaped article is chosen as a means for monitoring the attainment of a temperature of at least 70° C. in a particular implant, a skilled person will be able to select a suitable thermochromic pigment that is capable of displaying a colour change that reflects a temperature of at least 70° C. in the implant, such as thermochromic pigment that changes colour in a temperature range having a lower limit of a few degrees below 70° C.

The article can be made of any biocompatible or medical-device grade material that can be manufactured in a suitable form as described herein, and that can withstand the temperatures required to heat a prosthetic or implant as described herein. Preferably, the material should also be able to withstand the temperatures and other conditions that are customary during the sterilization, e.g. by autoclaving, of medical tools and devices. The material should be chemically inert and resistant to liquids and gases normally used in medical procedures, such as iodine and chlorhexidine solutions. Examples of suitable materials are, but not limited to, synthetic rubbers, resins and plastics such as silicone rubbers and resins, urethane resins, acrylonitrile butadiene rubber (NBR), chloroprene (neoprene), ethylene propylene diene monomer (EPDM), polyether block amide (PEBA), polyamide resins, polypropylene (PP) resins, polyethylene (PE) resins, polyphenylsulfone (PPSU or PPSF) and polyetheretherketone (PEEK) resins. Since all these materials typically are thermally insulating, the shaped article of the present disclosure favourably provides a thermal barrier for any surrounding tissue during heating, thus reducing the thermal dose to this surrounding tissue.

The articles can be manufactured with processes known in the art, and the skilled person will be aware of suitable manufacturing techniques based on the material chosen and the shape of the article to be provided. Examples of suitable techniques are extrusion, injection moulding, and 3D printing.

The shaped article according to the present disclosure may further comprise one or more coils at least partly embedded in said shaped article, wherein said one or more coils are configured to inductively heat at least a portion of the prosthetic or implant. By (partially) embedding the coils for inductive heating in the article itself, no external device is required for heating the prosthetic or implant. A further advantage is that embedding one or more induction coils in the article itself permits such coils to be maintained at a constant distance of the implant or prosthetic during induction heating. Furthermore, it allows for targeting only the portion of the prosthetic or implant to be heated, such as with a precision coil. In an alternative embodiment, the one or more coils may be used for cooling.

In one embodiment, the shaped article contains one induction coil embedded in said shaped article. In one embodiment, the shaped article contains two induction coil embedded in said shaped article. In one embodiment, the shaped article contains three or more induction coils embedded in said shaped article. The one or more coils are typically sized and dimensioned to be smaller than a body portion associated with the prosthetic or implant. For example, the coil may comprise a smaller diameter and longitudinal extent than a limb associated with the prosthetic or implant, e.g. a coil may have a smaller diameter than a diameter of a patient's leg. Preferably, the one or more coils are fully embedded in the shaped article. Such coils embedded in the shape allow precise control of the distance of the coil to the metal or metal part of the implant or prosthetic, keeping it secured (e.g., not moving) and allowing segmental heating of the implant or prosthetic by passing current through one (or more) coils or total heating when current is passed through all coils at once.

The one or more coils to inductively heat at least a portion of the prosthetic or implant by the application of Pulsed Electromagnetic Field (PEMF) with an operating frequency of less than about 100 kHz supplied by an apparatus connected to the one or more coils, wherein said apparatus is capable of controlling the power, such as an amount and/or duration of power, supplied to the one or more coils.

As an exemplary embodiment, there is provided a mould having a shape such that it tightly fits on a metal femoral component of a total knee replacement. The mould may be flexible to allow placement during a surgical procedure through a surgical incision. The mould may be made of silicon. The mould may comprise one or more induction coils configured for inductively heating the metal femoral component, which are partially of fully, preferably fully embedded in the body of the mould. The mould comprises means for monitoring a temperature change in said mould. The means for monitoring a temperature change in said mould may comprise one or more temperature sensors attached to or embedded in said mould. The means for monitoring a temperature change in said mould may comprise a thermochromic pigment, polymer, ink or dye embedded in or provided as a coating on said mould, or a combination thereof.

As another exemplary embodiment, there is provided a mould having a shape such that it tightly fits on a metal tibial component of a total knee replacement. The mould may be flexible to allow placement during a surgical procedure through a surgical incision. The mould may be made of silicon. The mould may comprise one or more induction coils, configured for inductively heating the metal tibial component, partially of fully, preferably fully embedded in the body of the mould. The mould comprises means for monitoring a temperature change in said mould. The means for monitoring a temperature change in said mould may comprise one or more temperature sensors attached to or embedded in said mould. The means for monitoring a temperature change in said mould may comprise a thermochromic pigment, polymer, ink or dye embedded in or provided as a coating on said mould, or a combination thereof.

As another exemplary embodiment, there is provided a flexible sheet having a shape such that it tightly covers the upper surface of a fracture fixation plate, for example a fracture fixation plate having length of 60 mm and width of 5 mm. The sheet is flexible to allow placement during a surgical procedure through a surgical incision. The sheet may be made of silicon. The sheet may comprise one or more induction coils, configured for inductively heating the fracture plate, partially of fully, preferably fully embedded in the body of the sheet. The sheet comprises means for monitoring a temperature change in said sheet. The means for monitoring a temperature change in said sheet may comprise one or more temperature sensors attached to or embedded in said sheet. The means for monitoring a temperature change in said sheet may comprise a thermochromic pigment, polymer, ink or dye embedded in or provided as a coating on said sheet, or a combination thereof.

As another exemplary embodiment, there is provided a mould having a shape such that it tightly covers the outer surface of a large trauma implant plate. The mould may be flexible to allow placement during a surgical procedure through a surgical incision. The mould may be shaped such that it allows (further) adapting or customising the shape during surgery. The mould may be made of silicon. The mould may comprise one or more induction coils, configured for inductively heating the metal tibial component, partially of fully, preferably fully embedded in the body of the mould. The mould comprises means for monitoring a temperature change in said mould. The means for monitoring a temperature change in said mould may comprise one or more temperature sensors attached to or embedded in said mould. The means for monitoring a temperature change in said mould may comprise a thermochromic pigment, polymer, ink or dye embedded in or provided as a coating on said mould, or a combination thereof.

According to a further aspect there is provided a method of inductively heating at least a portion of a prosthetic or implant, the method comprising
a) covering at least portion a prosthetic or implant with the shaped article as described herein; and
b) inductively heating said at least a portion of the prosthetic or implant using one or more induction coils.

The inductive heating of at least a portion of a prosthetic or implant may be achieved through one or more induction coils that form part of an external heating apparatus, or it may be achieved through one or more induction coils that are at least partially embedded in the shaped article as described above.

The method may comprises inductively heating the portion of the prosthetic or implant until a predetermined visual indication representing a predetermined temperature or temperature change has been reached in at least a portion of said article. Said visual indication representing a predetermined temperature or temperature change may be change in optical transmittance, a change in optical reflectance, a change in luminescence or a combination thereof in response to a temperature change, as described herein above.

The method may comprise treating an infection, such as a prosthetic joint infection (PJI). The method may comprise an infection treatment method. The method may comprise at least assisting in killing bacteria. The method may comprise heating bacteria to a critical temperature whereby bacteria are at least weakened, such as to assist in infection control by other means such as administered drugs and/or immuno-defence mechanisms.

Accordingly, the method may further comprise administering a drug, able to control infection, preferably a biocidal or biostatic drug, more preferably an antimicrobial composition effective in reducing the active count of isolated microorganism, at a concentration at the location of the prosthetic or implant suitable to achieve a reduction of the active count of microorganisms, to at least assist in treating an infection. Preferably, the antibiotic composition comprises at least one antibiotic compound selected from the family of antibiotics comprising penicillins, cephalosporins, aminoglycosides, tetracyclines, sulfonamides, macrolide antibiotics and/or quinolones, or the group comprising imipenem, aztreonam, chloramphenicol, erythromycin, clindamycin, spectinomycin, vancomycin, rifampin, bacitracin, methenamine, tobramycin, and nitrofurantoin. Preferably, the method may comprise administering the antimicrobial treatment at a dosage effective to produce a biocidal or biostatic, preferably bacteriostatic or fungistatic concentration at the biofilm site after the heating treatment. Alternatively, antimicrobial compositions, or other suitable compounds may be administered before the induction heating to enhance the effectiveness of the heat stress. Antimicrobial compositions and other compounds may also be used in between heating cycles to kill bacteria or slow down their growth in case multiple heating cycles are needed. The present invention hence also relates to a drug capable of controlling or removing an infection, preferably wherein the drug comprises a biocidal composition, preferably antimicrobial composition, for use of the treatment of infections in combination with a heat treatment according to the present invention, in patients in need thereof.

The method may comprise heating at least a portion of the prosthetic of implant in vitro prior to insertion or application of the portion of the prosthetic or implant to the patient. Additionally or alternatively, the method may comprise heating the portion of the prosthetic or implant in vivo, whilst the portion of the prosthetic or implant is attached to, or located in, the patient—or being attached to, or being located in the patient. In either case, at least a portion of the prosthetic or implant is covered with the shaped article according to the present disclosure. In either case, heating the at least a portion of the prosthetic of implant may be achieved through one or more induction coils that form part of an external heating apparatus, or it may be achieved through one or more induction coils that are at least partially embedded in the shaped article as described above.

In at least some examples, the method may comprise heating the portion of prosthetic or implant during a surgical procedure. The method may comprise an invasive surgical procedure. Alternatively, the method may comprise a non-invasive procedure. The method may comprise a treatment of the prosthetic or implant, but not a treatment of the patient as such.

The method may comprise in vivo use. The method may comprise heating the portion of the prosthetic or implant whilst the portion of the prosthetic or implant is located in and/or attached to a body. Additionally or alternatively, the method may comprise ex vivo use. The method may comprise in vitro use. The method may comprise heating the portion of the prosthetic or implant whilst the portion of the prosthetic or implant is located outside and/or detached from the body. In at least some examples, the method may comprise heating the portion of the prosthetic or implant ex vivo during a surgical procedure, such as where the prosthetic or implant (or portion thereof) is detached and/or removed from the body.

The method may comprise heating the portion of the prosthetic or implant during the surgical procedure without unduly heating patient tissue. The method may comprise heating the portion of the prosthetic or implant to a temperature at or below a maximum temperature. The maximum temperature may be associated with tissue damage. For example, the method may comprise heating the portion of the prosthetic or implant only to temperatures unassociated with undesirable tissue damage. The method may comprise preventing or at least minimising tissue damage due to the presence of means for monitoring a change of temperature in the shaped article that at least partially covers the prosthetic or implant during the procedure. By having such means embedded in, or attached to the shaped article according to the present disclosure, temperature measurement and/or temperature control of the prosthetic or implant is possible without damaging or scratching the prosthetic or implant itself. Furthermore, the presence of such a means to monitor a change of temperature in the object also allows for selectively heating a single portion of the prosthetic or implant at a time. Advantageously, the shaped article according to the present disclosure further provides a thermal barrier for any surrounding tissue during heating, thus reducing the thermal dose to this surrounding tissue.

The method may comprise heating the portion of the prosthetic or implant to a temperature at or below a minimum temperature. The minimum temperature may be associated with infection control. For example, the minimum temperature may be associated with killing, or at least combatting, infection. The method may comprise heating the portion of the prosthetic or implant to a temperature in a range, such as in a range from about 65° C. to about 70° C. The shaped article according to the present disclosure may provide a visual indication of whether a predetermined temperature or temperature change has been reached in at least a portion of said article.

The method may comprise heating the portion of the prosthetic or implant to a target temperature. The method may comprise controlling heating in dependence of a temperature of the portion of the prosthetic or implant, and/or additional temperature/s, such as of other portion/s of the prosthetic or implant and/or tissue (e.g. bone, muscle, cartilage, nerves, etc). The method may comprise controlling heating in dependence on a thermal sensor or thermometer. The method may comprise direct temperature measurement/s. The method may comprise operating a temperature measurement device. The method may comprise providing an augmented and/or a virtual reality. The method may comprise adapting the heating, such as via the power output to or from the coil, in dependence on the temperature measurement device. The method may comprise automatically adapting the heating. Additionally, or alternatively, the method may comprise manually adapting the heating. For example, the method may comprise an operator or user of the device manually adapting the heating in dependence on information the operator or user receives from the temperature measurement device. The method may comprise real time visualisation and/or measurement, such as provided by a display connected to or associated with an infra-red thermal camera. The method may comprise adapting the heating in dependence on a cumulative thermal dose. The method may comprise calculating the cumulative thermal dose real time. The method may comprise displaying the temperature/s and/or cumulative thermal dose/s to the user or operator, such as a surgeon. In at least some examples, the method may comprise automatically adapting the heating with an integral thermal sensor and a (micro-)controller comprised in the heating apparatus.

In an exemplary embodiment, during heating, only a portion of a prosthetic or implant is covered with the shaped article as described herein. Thus, the method may comprise heating only a portion of the prosthetic or implant. For example, the method may comprise not heating the entire prosthetic or implant. The method may comprise inducing heat in only a single portion of the prosthetic or implant at a time. The method may comprise not heating the entire prosthetic or implant simultaneously.

The method may comprise selectively heating only a portion of the prosthetic or implant. The method may comprise selectively heating only part of the implant that is safe. The method may comprise selectively heating only part of the implant that is no or low-risk. The method may comprise avoiding heating areas of the implant associated with bone fixation (e.g. for total joint replacements); and/or areas of the prosthetic or implant in proximity to or associated with important anatomical structures such as nerves (e.g. in case of infected plate in elbow). Alternatively, high risk areas (e.g. for loss of bone fixation or proximity of nerves/vessels) may be heated at or to a lower temperature/s (e.g. relative to low risk areas). The method may comprise targeting only the portion of the prosthetic or implant to be heated, such as with a precision coil. Such a precision coil may be an external coil, or it may be a coil embedded in the shaped article as disclosed herein.

The method may comprise inductively heating a first portion of the prosthetic or implant, whilst not inductively heating a second portion of the prosthetic or implant. The method may comprise allowing heat transfer from the first portion of the prosthetic or implant to the second portion of the prosthetic or implant. The method may comprise utilising the second portion of the prosthetic or implant as a heat sink for the first portion of the prosthetic or implant. Additionally or alternatively, the method may comprise cooling, such as actively cooling, the coil; and/or the portion of the prosthetic and/or other portion/s of the prosthetic or implant; and/or patient tissue.

The method may comprise sequentially heating two or more portions of the prosthetic or implant, such as by heating a first portion prior to heating a second portion of the prosthetic or implant.

In at least some examples, the method comprises heating the entire prosthetic or implant.

The invention will now be described in detail with reference to the non-limiting embodiments shown in the Figures. The subject matter that is presently disclosed is not limited to the embodiments only, but also includes every other combination of features described herein or recited in mutually different dependent claims.

### Detailed Description of the Drawings

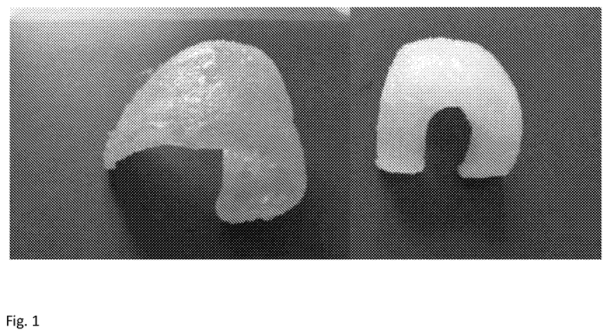

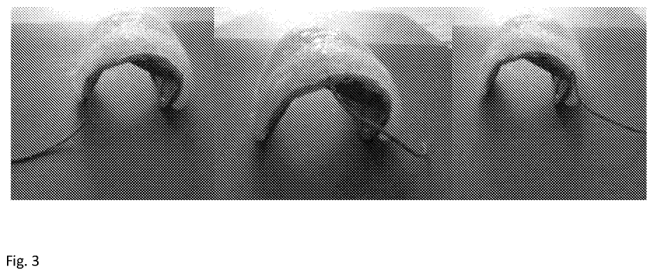

FIG. 1 shows an example of a shaped article in frontal and sidewise view. The article is made of silicon shaped such that it fits on a metal femoral component of a total knee replacement (see also FIG. 3). The shaped article is flexible to allow placement during a surgical procedure through a surgical incision. The article does not contain means for monitoring a temperature change in the article.

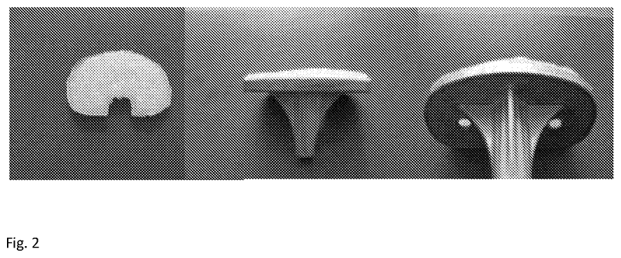

FIG. 2 (left) shows an example of a shaped article. The article is made of silicon shaped such that it fits on a metal tibial component of a total knee replacement (middle and right). The shaped article is flexible to allow placement during a surgical procedure through a surgical incision. The article does not contain means for monitoring a temperature change in the article.

FIG. 3 shows various views of a shaped article according to an embodiment of the present disclosure. The article is made of flexible silicon having a shape as shown in FIG. 1, for fitting around a femoral component. The article keeps a wire with a thermal sensor (thermocouple) fixed between the shaped article and the femoral implant, thus allowing temperature measurement of the surface of the metal femoral component. The shaped article allows fitting of multiple thermal sensors within the silicon shape at the surface of the article, touching the surface of the metal implant and thus allowing temperature measurements of multiple locations.

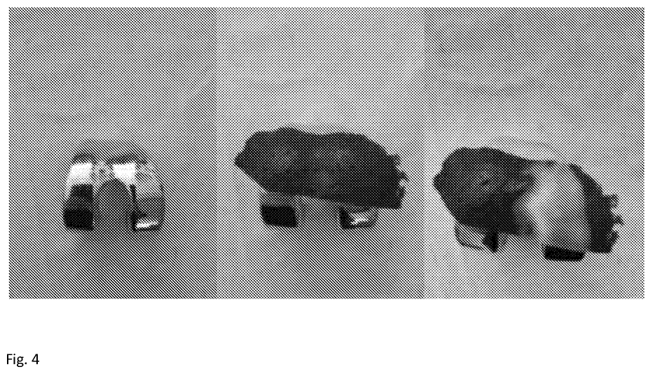

FIG. 4 shows a metal femoral component (left image) of a total knee replacement partially covered (centre and right image) with a shaped article made of silicon mixed with a thermochromic compound that changes colour from dark green to light green/white upon heating to about 32° C. Before heating (centre image), both sides (femoral condyles) are dark green. After induction heating of one of the femoral condyles, the part of the article covering the heated condyle has turned white, thus providing a visual indication of the temperature change and signalling that the target temperature (as related to the thermochromic compound) has been reached.

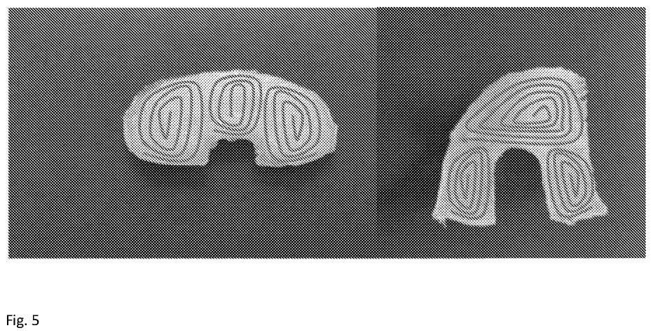

FIG. 5 shows exemplary shapes for tibial and femoral articles with possible arrangements of induction coils (drawn). Such coils can be embedded in the shape allowing precise control of the distance of the coil to the metal implant, keeping it secured (not moving) and allowing segmental heating of the implant by passing current through one (or more) coils or total heating when current is passed through all coils at once.

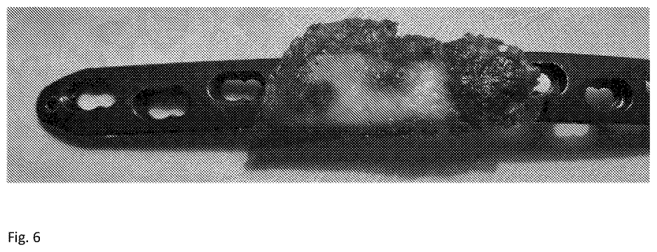

FIG. 6 shows a metal fracture plate partially covered with a shaped article made of silicon mixed with a thermochromic compound that changes colour from dark green to light green/white upon heating to about 32° C. The heated part of the plate is visible as being white. The dark spots in the white area are due to the holes in the plate below the shaped article, under which there is no heated metal.

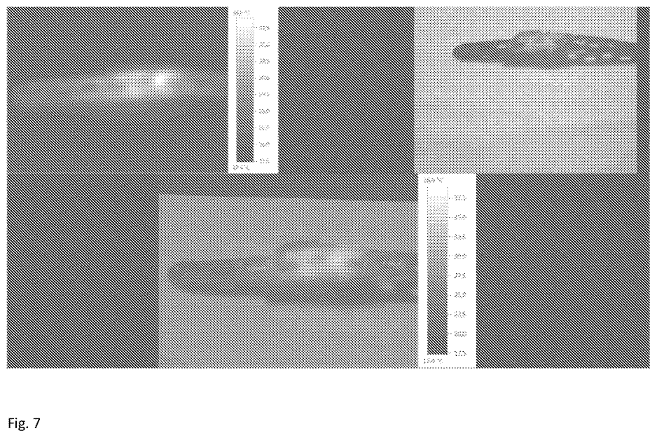

FIG. 7 shows a thermal image (top left), a normal photograph (top right) and a merged image (bottom) of a metal (stainless steel) fracture plate partially covered with a shaped article made of silicon mixed with a thermochromic compound that in this case changes colour from dark green to light green/white upon heating to about 32° C. The merged image shows that the hottest parts of the implant (more than 31° C.) correspond with the white area.

1. A shaped article sized and dimensioned to at least partly and removably cover a prosthetic or implant, wherein said shaped article comprises means for monitoring a temperature change in said article.

2. The shaped article according to claim 1, wherein said means for monitoring a temperature change in said article comprises one or more temperature sensors attached to or embedded in said shaped article.

3. The shaped article according to claim 1, wherein said means for monitoring a temperature change in said article comprises a material that is capable of providing a visual indication of whether a predetermined temperature or temperature change has been reached in at least a portion of said article.

4. The shaped article according to claim 1, wherein said material that is capable of providing said visual indication comprises a material that exhibits a change in optical transmittance, a change in optical reflectance, a change in luminescence, or a combination thereof in response to a temperature change.

5. The shaped article according to claim 4, wherein said material that displays a change in optical transmittance, a change in optical reflectance, a change in luminescence, or a combination thereof is a thermochromic pigment, thermochromic polymer, thermochromic ink, or thermochromic dye; and
said material that displays a change in optical transmittance, a change in optical reflectance, a change in luminescence, or a combination thereof is embedded in, provided as a coating on, or a combination thereof, said shaped article.

6. The shaped article according to claim 3, wherein said change in optical transmittance, change in optical reflectance, change in luminescence, or a combination thereof in response to a temperature change is a reversible change, an irreversible change, or a combination thereof.

7. The shaped article according to claim 1, wherein the body of said shaped article is made of a biocompatible plastic or rubber.

8. The shaped article according to claim 1, further comprising one or more coils at least partly embedded in said shaped article,
wherein said one or more coils are configured to inductively heat at least a portion of the prosthetic or implant.

9. The shaped article according to claim 1, wherein said article is sized and dimensioned to generically at least partly cover multiple shape variants within a class of prostheses or implants.

10. The shaped article according to claim 1, wherein said article is sized and dimensioned to generically at least partly cover an implant or prosthetic selected from the group consisting of:
a tibial component for knee replacement surgery, a femoral component for replacement surgery, a tibial component for ankle replacement surgery, a talar component for ankle replacement surgery, a glenoid component for shoulder replacement surgery, a humeral (stem) component for shoulder replacement surgery, an acetabular component for hip replacement surgery, a femoral stem implant component for hip replacement surgery, and a fixation device for clavicle, spine, ankle, hip, tibia, or femur fractures.

11. The shaped article according to claim 1, wherein said shaped article is in the form of a flexible sheet.

12. The shaped article according to claim 1, wherein said shaped article is dimensioned to substantially follow the contours of an external surface of the prosthetic or implant.

13. The shaped article according to claim 1, wherein said shaped article comprises one or more induction coils configured for inductively heating at least a portion of said shaped article.

14. The shaped article according to claim 1, wherein said shaped article comprises one or more temperature sensors attached to or embedded in said shaped article.

15. A method of inductively heating at least a portion of a prosthetic or implant, the method comprising
a) covering at least portion a prosthetic or implant with the shaped article according to claim 1; and
b) inductively heating said at least a portion of the prosthetic or implant using one or more induction coils.

16. The method according to claim 15, wherein the inductively heating of at least a portion of the prosthetic or implant using one or more induction coils occurs via one or more induction coils embedded in the shaped article.

17. The method according to claim 15, wherein the method comprises inductively heating the portion of the prosthetic or implant until a predetermined visual indication representing a predetermined temperature or temperature change has been reached in at least a portion of said article.

18. The method according to claim 15, wherein the method further comprises administering a biocidal composition effective in reducing the count of harmful microorganisms at a concentration at the location of the prosthetic or implant suitable to achieve a reduction of the count of microorganisms.

19. The shaped article according to claim 11, wherein said flexible sheet can be further modelled and/or cut into a desired shape.

20. The method according to claim 18, wherein the biocidal composition is an antimicrobial composition.
